(19) United States
(12) Patent Application Publication
(10) Pub. No.: US 2010/0114080 A1
(43) Pub. Date: May 6, 2010

(54) APPARATUS, SYSTEM AND METHOD FOR MEDICAL TREATMENT

(76) Inventor: Richard H. Theriault, Lincoln, MA (US)

Correspondence Address: LANDO & ANASTASI, LLP, ONE MAIN STREET, SUITE 1100, CAMBRIDGE, MA, 02142, US

(21) Appl. No.: 12/612,887

(22) Filed: Nov. 5, 2009

Related U.S. Application Data
(60) Provisional application No. 61/111,492, filed on Nov. 5, 2008.

(51) Int. Cl. A61B 18/18; G06Q 10/00; G06Q 50/00

(52) U.S. Cl. 606/9; 705/300; 705/3

(57) ABSTRACT

According to one aspect, a system includes an energy application device configured to apply energy to a treatment area of a subject, a sensing device configured to determine the energy received by one or more regions of the treatment area, and a user interface configured to provide a user with feedback concerning the energy received by the one or more regions of the treatment area, wherein the feedback provided to the user includes an image that illustrates the current status of the treatment. According to one embodiment, the illustration includes quantitative information. According to a further embodiment, the illustration includes highlighting of the treatment area.

### RELATED APPLICATIONS

This application claims the benefit under 35 U.S.C. §119(e) to U.S. Provisional Application Ser. No. 61/111,492, entitled “APPARATUS, SYSTEMS AND METHODS FOR MEDICAL TREATMENT,” filed on Nov. 5, 2008 which is herein incorporated by reference in its entirety.

### BACKGROUND OF INVENTION

Historically, the treatment of skin conditions was handled by dermatologists. Today, however, an increasing percentage of such treatment is performed by physicians and internists who are qualified, but not as well versed in the treatments as dermatologists because that is not their specialty.

In addition, the practice of medicine has shifted to a more traditional business model with a greater focus on profit and loss. As a result, marketing has become relevant to the practice of medicine.

Further, manufacturers and distributors of medical devices and systems provide these devices and systems but generally do not closely track the application of the device or system nor whether patients might be better served with treatments employing other devices or systems which can also be supplied by the manufacturer. As a result, there is a disconnect between the available treatment at a medical office and other treatment approaches which may be available for patients because the physician is not familiar with their availability and/or application.

### SUMMARY OF INVENTION

Therefore, there is a need for systems that facilitate the medical treatment (for example, aesthetic medical treatment) by those who do not specialize in the field. The systems may provide greater feedback and guidance regarding the application of the medical treatment, the status of the treatment protocol and recommendations for further and/or different treatments to be included in the protocol. The recommendations can be the result of any of the status of the current treatment protocol, the patient's present condition including conditions resulting from the current treatment, the treatment goals and the results of possible future treatments in view of past results with similarly situated patients.

According to one aspect, a system includes an energy application device configured to apply energy to a treatment area of a subject, a sensing device configured to determine the energy received by one or more regions of the treatment area, and a user interface configured to provide a user with feedback concerning the energy received by the one or more regions of the treatment area, wherein the feedback provided to the user includes an image that illustrates the current status of the treatment. According to one embodiment, the illustration includes quantitative information. According to a further embodiment, the illustration includes highlighting of the treatment area.

According to another aspect, a computer-based method includes acts of: storing patient treatment data for a subject patient, storing reference treatment data for a plurality of patients where the reference treatment data includes at least one image, evaluating a current status of treatment for the subject patient, and providing at least one recommended course of treatment for the subject patient based on a comparison between the current status and a result achieved in treating a similarly situated patient. In some embodiments, the computer-based method includes an act of including at least one image of the similarly situated patient with the recommendation. In a further embodiment, the recommendation is communicated to the patient via the Internet. According to one embodiment, the computer-based method is a method of evaluating a treatment of an aesthetic condition of the subject patient.

According to yet another aspect, a computer-based method includes acts of: centrally-collecting information concerning patient treatment from a plurality of practices where the information concerns recommended courses of treatment and associated results; storing information concerning a plurality of products and services available from a supplier of treatment systems; receiving at least one practitioner recommended treatment for a subject patient, evaluating the recommended treatment in view of (a) the current treatment status of the subject patient, (b) centrally collected information concerning at least one similarly situated patient and (c) the plurality of products and services available from the supplier; and providing at least one recommended change to the practitioner recommended treatment based on the act of evaluating.

In another aspect, the invention provides a computer-based method of enhancing at least one treatment of an aesthetic condition of patients, the method including acts of collecting and evaluating patient treatment histories from a plurality of practices for the aesthetic condition against results available from a set of products and services provided by a supplier and generating at least one recommended product or service based on a result of the evaluation. In one embodiment, the recommendation is automatically generated and communicated to at least one participating practice.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a system in accordance with an embodiment of the invention;

FIG. 2 illustrates a system in accordance with another embodiment of the invention; and

FIG. 3 illustrates a system in accordance with yet another embodiment of the invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

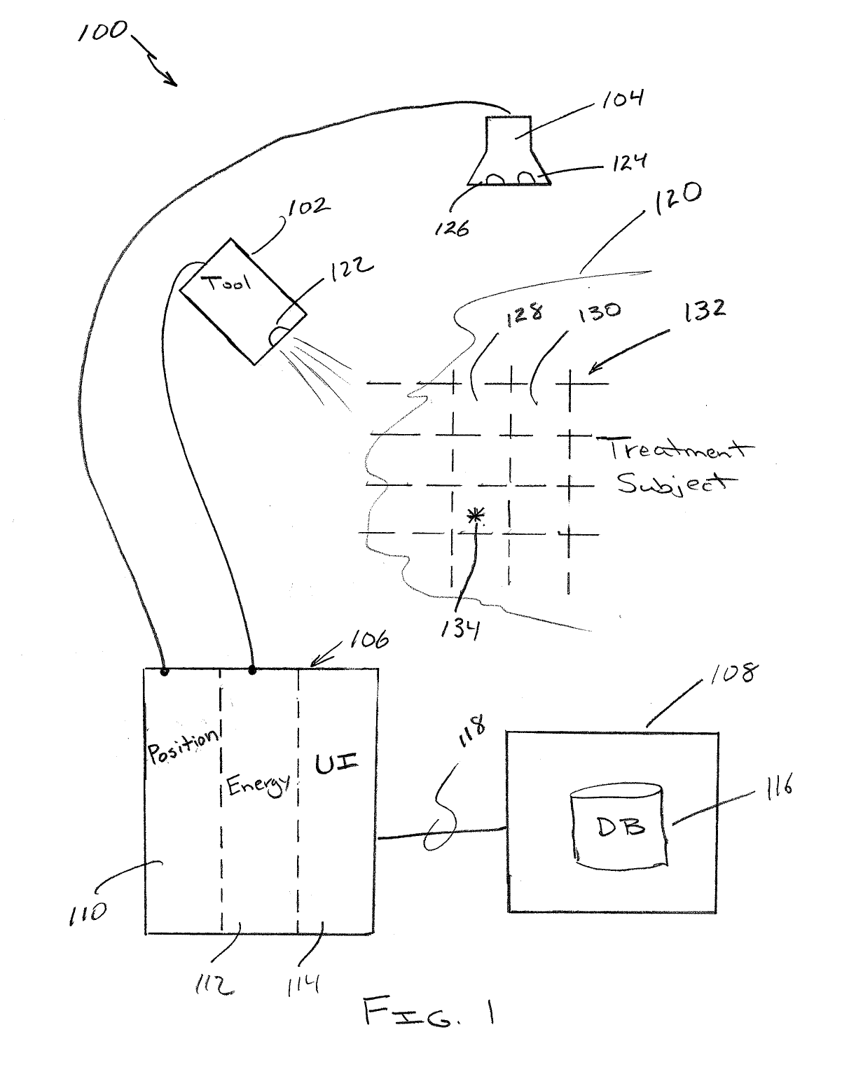

Referring to FIG. 1, the system 100 for medical treatment is illustrated. In accordance with various embodiments, the system is directed to an aesthetic medical treatment procedure, such as hair removal, blemish removal, and tattoo removal. The system 100 includes an energy application device 102, a coverage sensing device 104, a control system 106, and a computer system 108. In accordance with the illustrated embodiment, the control system 106 includes a sensing module 110, an energy application module 112, and a user interface 114. In a further embodiment, the computer system 108 includes a database 116. In accordance with one embodiment, the control system 106 and the computer system 108 communicate with one another over a network 118. In general, the system 100 applies energy to achieve a desired treatment objective for a subject 120.

According to one embodiment, the energy application device 102 includes an energy output 122. The energy output can generate a light output (for example, a laser output, a UV output), a mechanical output, ultrasound, and various other forms of RF energy. In accordance with some embodiments, the energy application device 102 is a hand-held unit. For example, a hand-held wand that a practitioner can move across regions of a patient, i.e., across a treatment area. In accordance with other embodiments, the energy application device is part of a larger device that is moved by a mechanical means, for example, by a robotic means to apply the desired energy to the subject 120.

In accordance with the illustrated embodiment, the coverage sensing device 104 may include a device for tracking the application of energy to the subject 120. For example, the device 104 may include a receiving element 124 such as a camera that is employed to capture images of the treatment area. In accordance with another embodiment, the coverage sensing device may include a signal generating means 126 that is applied to the treatment area to generate information that is sensed by the receiving element 124. For example, the signal generating element 126 may be a light source, for example, a UV light source and the receiving element may include a receiver to receive selected wavelengths of reflected light.

In the illustrated embodiment, the energy application device 102 is connected to the energy application module 112. In accordance with one embodiment, the energy application module 112 controls the energy output 122 of the energy application device 102, for example, the intensity of the energy supplied at the energy output. In accordance with a further embodiment, the sensing module 110 receives information from the coverage sensing device 104. In a further embodiment, energy application is controlled using controls included in the energy application device 102.

According to one embodiment, a treatment area of the subject 120 may be associated with one or more subregions, for example, a first subregion 128 and a second subregion 130. These subregions 128 and 130 may be included as part of a coordinate system 132. In the illustrated embodiment, the system 132 is a grid, however, the coordinate system need not include parallelograms and may instead include any geometric shape or plurality of geometric shapes that will allow the system 100 to identify a location within the treatment area. In some embodiments, the coordinate system 132 allows the system 100 to correlate the application of energy by the energy application device 102 to a localized portion of the treatment region so that the progress of the energy application can be determined. In some embodiments, the system 100 tracks the progress of the treatment to provide a user with a degree of completion of the treatment. Further, the coordinate system 132 need not include any geometric shape and instead may simply include one or more fiducials 134 which may be used as markers to identify a particular location on the treatment area from which the position of the energy application device 102 can be determined. In one embodiment, one or more fiducials are employed in combination with a coordinate system. Further, in some embodiments, the coordinate system includes information concerning the topography of the treatment area. In some embodiments, the preceding approaches provide a user with information from which they can adjust the treatment (for example, adjust the treatment in real-time). That is, the user can adjust the intensity, area of coverage and/or duration of the treatment based on the degree of completion. In any of the above embodiments, the coordinate system 132 can be overlayed the treatment area and presented in the display for reference.

In accordance with some embodiments, the energy application module 112 controls the energy provided at the energy output 122 based on one or more patient parameters and/or one or more treatment parameters. In accordance with one embodiment, patient parameters can include the physical body part where the treatment area is located, the skin type of the patient (coloration, melanin, etc.), the elasticity of the skin of the patient, the dryness of the skin of the patient, and the patient's age. In some embodiments, the treatment parameters can include parameters related to the energy application and the energy source. For example, the treatment parameters may include a relaxation time between energy deliveries, a selected combination of energy sources to be applied in a treatment session, the energy intensity applied during each pass of the energy application device 102, and the rate of travel of the energy application device 102 over the treatment area. In accordance with various embodiments, this information is supplied to the control system 106, and in particular, the energy application module 112 which employs the information to adjust the energy output of the energy application device 102. According to one embodiment, the output control is performed automatically by the control system 106.

In some embodiments, the user interface 114 allows the practitioner or other medical personnel to provide one or more of the treatment parameters and/or the patient parameters to the control system 106. In a further embodiment, the computer system 108 may also provide input employed by the control system 106 when it determines the correct energy level for the treatment.

According to some embodiments, the energy application device 102 and the coverage sensing device 104 are included in a single device, for example, a hand-held device. In one embodiment, the energy application device and the coverage sensing device are included in the single hand-held device which includes a camera as the receiving element 124.

According to some embodiments, the user interface 114 includes a display. In accordance with one embodiment, the display is used to display an image of the treatment area in substantially real time to the practitioner who is performing the treatment. In some embodiments, the display provides feedback in substantially real time concerning the amount of energy applied and in some cases, the progress of the treatment relative to the objective for that treatment session. For example, the treatment area may be broken down into coverage areas, for example, the subregions 128 and 130 and those regions may be shaded according to the progress of the treatment in that treatment session. As a result, the practitioner is supplied with feedback concerning the progress of the treatment for the session and can adjust the application of energy to the treatment area to ensure that the full treatment area has received substantially the correct energy dose. The information provided in the display included in the user interface 114 is quantitative information in one embodiment. For example, the information that is provided may indicate the actual energy applied in that treatment session.

In addition, the display may include cumulative data that indicates the energy received by the treatment area or regions within the treatment area for a treatment protocol that employs more than one treatment session. In accordance with other embodiments, the information may be provided in a more simplified non-quantitative format that allows a practitioner to quickly assess whether the energy levels applied meet the objectives of the treatment session and/or pose any risk to the treated region. For example, a color-coded scheme may be employed. According to one embodiment, a green coloration indicates to the practitioner that the goals for the treatment session are not met with the energy received thus far during the treatment. Accordingly, the practitioner would know they should continue to treat that region of the treatment area. A second coloration may indicate that the treatment levels are approaching the treatment goals (based on the energy applied) for that treatment session and/or that a treatment protocol that spans more than one treatment session. A further different coloration (different color, different shade, different intensity, etc.) may indicate that the practitioner is approaching or has exceeded the recommended coverage levels or energy intensity for the region of the treatment area either or both for that treatment session or the treatment protocol spanning more than one treatment session.

As used herein, the term “treatment protocol” refers to a course of treatment which is intended to achieve a desired treatment objective. Accordingly, a treatment protocol can include a single treatment session or a plurality of treatment sessions. In addition, a treatment protocol can include treatment with a single type of energy or a plurality of types of energy applied to the treatment area.

In accordance with some embodiments, the computer system 108 provides one or more inputs concerning the patient parameters and/or the treatment parameters that are employed by the energy application module 112 to control the output of the energy application device 102.

According to some embodiments, the treatment area is the skin of the subject. According to one embodiment, the display includes identifies the areas that have received at least some treatment. In a further embodiment, the display provides the amount of energy applied per section of treatment area. In some embodiments, the display includes a topographic plot of energy level applied per unit skin area. In accordance with another embodiment, the display provides a nominal energy of one to ten per unit area of skin. Other visual identifiers per unit skin area may be employed.

In accordance with one embodiment, the computer system receives data from the control system which is stored in the database 116 for future reference. For example, the stored data may be referred to when further proposed treatments are assessed. The data may also be employed in another embodiment to adjust a preexisting recommended course of treatment. In yet another embodiment, the data may be stored for use in assessing the treatment of another patient who may be similarly situated (i.e., who may have one or more of the same skin conditions) as the patient for whom the data was collected. The data storage may include the storage of image data, for example, images of the treatment area. These may include progress images that show the treatment area at various stages of treatment which may provide a clear representation of the results achieved thus far in a treatment protocol. In addition, these images may provide data that can be used to present to other potential patients or current patients when recommending further treatments and/or changes to the treatment protocol for that patient. As indicated previously, the coverage sensing device 104 may include a receiving element 124 that includes a camera. In accordance with one embodiment, the camera is a video camera. Further, the video camera may continuously record video for viewing by the practitioner during the treatment procedure. With the feedback provided by the location of the hand piece to the control system, the practitioner can be provided information that allows them to continuously track the progress of the treatment with the video information.

The stored data can include data concerning the amount of energy that was received by the treatment area (including specific regions within the treatment area) in a single treatment session and/or the cumulative energy received in a series of treatments.

Various embodiments of the invention may be employed in aesthetic medicine generally. For example, in one embodiment, the system 100 may be employed in hair removal, while in another embodiment, the system 100 may be employed for the removal of blemishes or other pigment varying treatments.

In accordance with some embodiments, the network 118 includes a local area network (LAN). In accordance with another embodiment network 118 includes a wide area network (WAN). In accordance with still a further embodiment, the network 118 includes each of a LAN and a WAN.

According to various embodiments, the computer system 108 includes processing and memory components that can be employed to save and/or generate recommended treatments and treatment protocols.

According to one embodiment, the control system communicates one or more images captured by the coverage sensing device to the computer system 108, for example, via the network 118.

According to some embodiments, the energy application device 102 and the coverage sensing device 104 (whether separate devices or integrated into a single device) are hardwired to the control system 106. In accordance with another embodiment, the energy application device 102 and/or the coverage sensing device are wireless devices that communicate to the control system 106 and/or the computer system 108 wirelessly. According to one embodiment, all or a portion of the control system 106 is integrated in a handheld device in combination with the energy application device 102 and the coverage sensing device 104. In accordance with this embodiment, the handheld device communicates wirelessly with the computer system 108.

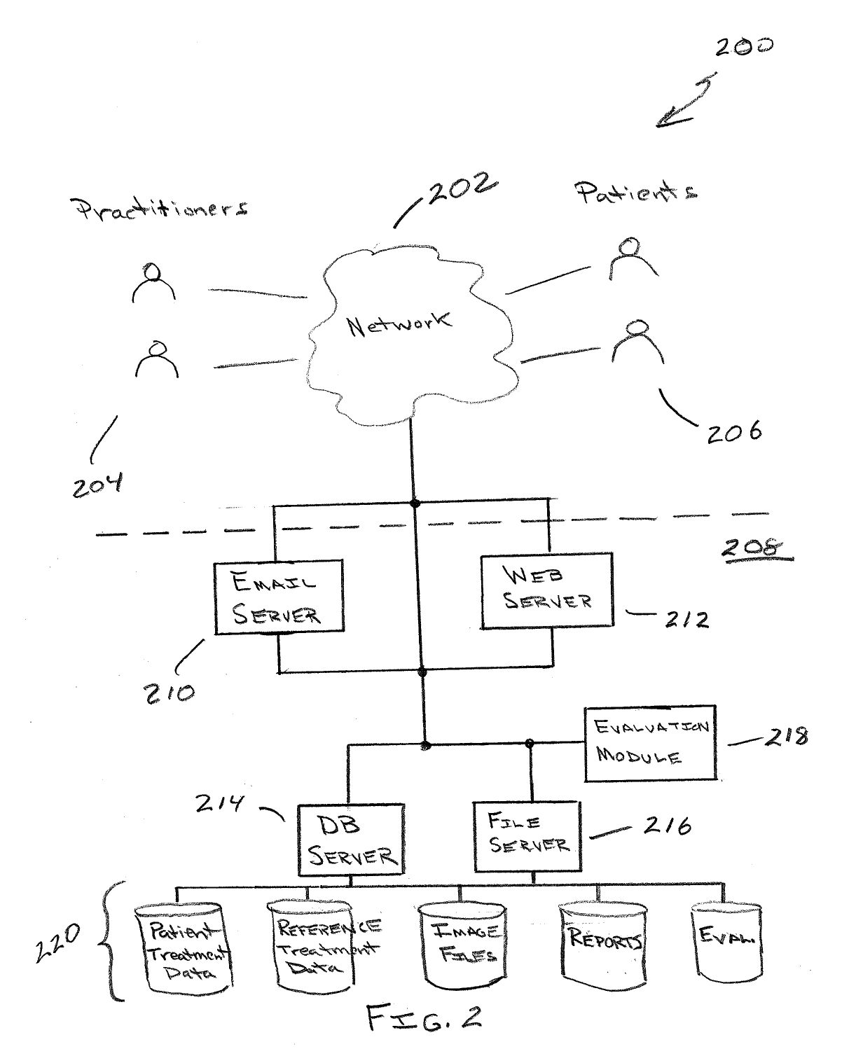

Referring now to FIG. 2, a computer-based system 200 is employed in one embodiment to provide practitioners and patients with information helpful in determining whether a treatment protocol should be undertaken. According to one embodiment, the system provides practitioners with information needed by them to make recommendations for treatments to patients. In further embodiments, the system can automatically generate reports for further treatments which can be communicated directly to the practitioners and the patients.

The system 200 includes a network 202 which connects a plurality of practitioners 204 and a plurality of patients 206 to a computer-based system 208. In accordance with one embodiment, the computer-based system 208 includes an e-mail server 210, a web server 212, a database server 214, a file server 216 and an evaluation module 218.

The computer-based system 208 can also include a plurality of sources of data 220 which may include patient treatment data, reference treatment data, image files, reports and other files that are stored in the computer-based system 208.

In accordance with some embodiments, the computer-based system 208 provides communication of the current treatment status to both patients and practitioners. Thus, according to one embodiment, the computer-based system 208 can also allow practitioners to generate reports and information concerning the patient's treatment for communication directly to anyone of the plurality of patients 206.

According to some embodiments, the computer-based system 208 can also allow practitioners to solicit patients to undergo treatment protocols based on information provided by the computer-based system 208 in various of these embodiments, the computer-based system 208 can also allow practitioners 204 to solicit new patients from among the plurality of patients 206.

Communication using a computer-based system may employ e-mail which can be communicated over the network 202 from the e-mail server 210. Information may also be communicated to practitioners 204 and patients 206 via a web server 212 where the internet is used. The communication may also include links to file data that can be provided by the file server 216 and other stored data that can be provided by the database server 214. As can be seen from the illustrated embodiment, the communication of information is facilitated by the computer-based system 208 because it employs a variety of communication means to communicate any of a variety types of data.

In accordance with some embodiments, patient treatment data is communicated to any one of a plurality of practitioners 204 so that a practitioner can evaluate the patient's current status in view of the treatment protocol. In evaluating the patient's condition, the practitioner can rely on information recorded in the prior treatment session, for example, one or more images which were collected of the treatment area during one or more of the prior treatment sessions. A practitioner may employ this information to evaluate how next to proceed for a given treatment protocol. According to some embodiments, the practitioner can review such information and determine that a different treatment protocol is more appropriate.

Other information which any of the practitioners 204 may refer to can include not only image files but other documents such as reports previously generated for the patient undergoing the treatment. These reports may include the status or past status of a treatment. The information may also include video information that may have been recorded during one or more past treatment sessions, for example, video of the treatment area while undergoing a prior treatment.

The computer-based system allows practitioners to provide the treatment information directly to anyone of the plurality of patients 206 via the network 202 in an easy-to-use electronic form. This may include e-mail communication to any one of the plurality of patients 206 and these e-mail communications may include links to HTML documents and can also include attachments, for example, attachments to PDFs or word processing documents including image files and reports.

In addition, the computer-based system 208 can also allow any of the plurality of practitioners 204 to communicate electronically with any of the plurality of practitioners 204. For example, a first practitioner among the plurality of practitioners 204 may contact a second practitioner to consult on a particular patient treatment. Such communication may be employed to evaluate whether further treatment might be beneficial and what particular treatments might be appropriate given the patient's current status. Thus, the electronic communication allows the practitioners to communicate information to one another easily and in a form that maximizes the amount of information that can be shared. For example, these communications may also include e-mails with attachments to various types of files that would be useful, such as the current status of a treatment and how a recommended treatment should proceed. Again the e-mails may also include links to web-based documents or web-based interfaces that require a practitioner to login to a secure system to access the information referred in the communication.

In some embodiments, the computer-based system 208 is a secure system that includes network access security as is well known but those of ordinary skill in the art. For example, the web server may provide to any of the practitioners 204 or patients 206 a login screen before allowing access to the types of information 220 that is maintained by the computer-based system 208. In accordance with one embodiment, the computer-based system 208 generates e-mail which is sent to a selected one or group of practitioners 204 and/or patients 206 where the e-mail server indicates that information is available to the practitioner and/or patient when they login into the computer-based system 208 using a web-based interface. Accordingly, the e-mail may include a link to an HTML interface to allow the practitioner 204 or patient 206 to login into the computer-based system to access the information referred to in the e-mail alert.

According to the illustrated embodiment, the stored information 220 may include reference treatment data. This reference treatment data in some embodiments can be employed to optimize a patient's treatment by providing a basis for comparison of the patient's current condition with the condition of one or more similarly situated patients for whom information is stored in the reference treatment data. In accordance to some embodiments, the reference treatment data may include any of image files and reports that were previously prepared based on the prior patient's treatment. In accordance with some embodiments, the evaluation module 218 of the computer-based system is employed to generate an output based on a comparison between the patient treatment data and the reference treatment data where the patient treatment data concerns a current patient for whom a recommendation is generated. In accordance with one embodiment, the computer-based system 208 provides the results of the comparison by the evaluation module and automatically forwards them to one or more selected practitioners 204 who are evaluating possible treatment for a selected patient. In accordance with another embodiment, the computer-based system 208 alerts the practitioner that such an evaluation is complete and available when they access the computer-based system 208. For example, the evaluations generated by the evaluation module may be stored with the stored information 220 of the computer-based system 208.

In some embodiments, a practitioner can review the data provided by the evaluation module 218 and make a determination about a course of treatment that would be best suited for the selected patient. According to one embodiment, the practitioner can review such a recommended course of treatment that is automatically generated by the computer-based system 208. In other embodiments, the practitioner can review and modify the recommended course of treatment provided by the computer-based system 208 and store the revised recommended course of treatment on the computer-based system 208. According to one embodiment, this recommended course of treatment can be communicated electronically over the network 202 by the computer-based system 208 to a selected one of the plurality of patients 206.

In a further embodiment, the recommendations generated by the evaluation module 218 may be communicated automatically to a patient to whom the recommendations apply. Thus, an e-mail alert can be generated by the computer-based system to notify the patient that a recommended treatment is available when they log into the computer-based system 208. In another embodiment an e-mail may also include an attachment of one or more files with the recommended treatment based on the output of the evaluation module 218.

In accordance with some embodiments, the computer-based system provides a tool for practitioners 204 to encourage patients 206 to continue their treatment. That is, a patient can be motivated to further their course of treatment when they are provided with relevant comparison information which may include a comparison of their current condition, a current condition of a similarly situated patient and the results achieved when the similarly situated patient underwent further treatments. In one embodiment, the further treatments are consistent with the treatment recommended for the current patient. Accordingly, the information provided to the patient 206 may include graphical information including image data that illustrates the improvement, for example, in an aesthetic treatment provided to a similarly situated prior patient. In one embodiment, the information concerning the prior patient is available via the stored information 220 such as reference treatment data and/or information image files concerning that patient.

Due to privacy laws and the sensitivity of sharing medical information the computer-based system 208 can include approaches to limit the disclosure of information concerning prior patients to third parties such as the current patient. This can be implemented in a variety of ways, for example, using databases and/or networks protected using cryptographic methods. In accordance with one embodiment the computer-based system 208 may be included with the control system 106 in a computer system 108.

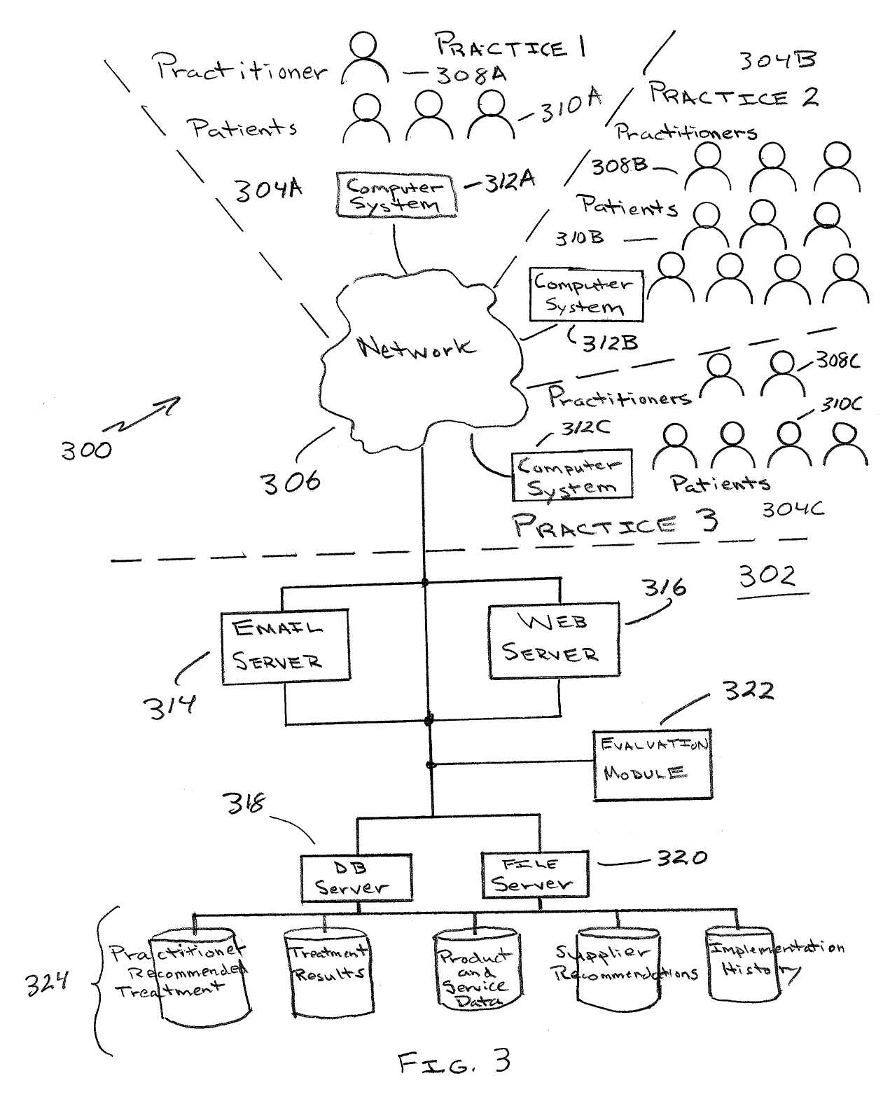

Referring now to FIG. 3 a system 300 is illustrated. System 300 includes a computer-based system 302 which is connected to a plurality of medical practices 304 via network 306. Each of the plurality of practices 304 may include one or a plurality of practitioners 308 and one or a plurality of patients 310. In addition, each practice may include one or a plurality of computer systems 312. The computer system 312 can include desktop, laptop computers and servers configured in a manner well known to those of ordinary skill in the computers art. The computer systems 312 may be configured in any manner that allows the storage and retrieval of patient records and patient treatment histories that are either stored directly in the computer system 312 or accessed from a remote resource via the network at 306.

In accordance with some embodiments the computer-based system 302 includes an e-mail server 314, a web server 316, a data based server 318, a file server 320 and an evaluation module 322. In accordance with a further embodiment, the computer-based system 322 includes stored information 324 can include information concerning a suite of products and/or services that are available through a supplier, supplier recommendations to practitioners concerning available products and services that may assist the practitioners practice, a history of practitioner recommended treatments associated with one or more of the patients 310, the treatment results concerning one or more patients 310 and an implementation history concerning the response by practitioners to supplier recommendations and further, the history concerning the patient treatment following an adoption of one or more supplier recommendations.

As a result, the system 300 can provide a supplier of medical products and/or services, in particular products or services directed to aesthetic medical treatment, with a system to monitor ongoing practitioner practices to generate recommendations for improved treatment results using a supplier's product and/or services.

In addition, the system 300 can provide practitioners with best practices developed using data from their own practice, and in addition, the practices of others. Thus, the system 300 allows a practitioner 308 to leverage the knowledge gained from the treatment provided by other practitioners in other practices to patients that the first practitioner is not even treating.

In accordance with some embodiments the computer-based system 302 provides a central repository for treatment information concerning multiple practices (and patients) which are unrelated to one another. Further, a computer-based system 302 can employ one or more security measures to ensure that protected and sensitive patient information is not available to unassociated practitioners and patients without prior authorization. These security measures can include access restrictions that require users provide some form of authentication such as a user name and password to access confidential patient information. In some embodiments, the authentication process includes encryption of confidential information, public-key protocols are used in one embodiment.

In accordance with some embodiments a computer-based system 302 includes the evaluation module 322 which is employed to perform one or more comparisons to determine whether practitioner recommended treatments can be improved to optimize the treatment results. For example, where the treatment is an aesthetic medical treatment to a skin surface, the computer-based system 302 can determine the effectiveness based on an improvement of appearance of the treated area under one or more practitioner recommended courses of treatment. According to one embodiment, the evaluation module 322 employs information concerning the practitioner recommended treatment and the treatment results to evaluate the success of that course of treatment with the first patient. In addition the evaluation module may also employ information concerning the suite of products and services available and their capabilities to determine whether the recommended course of treatment can be further improved by using a selected one or more of the available products and services.

In accordance with some embodiments, the computer-based system 302 although being centralized includes a more limited subset of information which is employed in combination with information that is selectively released by the practices based on their practice specific criteria. That is, a particular practice and/or practitioner may opt to maintain the full records concerning the practitioner recommended treatments and treatment results on their own computer system 312 and offer access by the computer-based system 302 to a limited subset of that information, for example, upon request by the practice for one or more supplier recommendations in a specific instance. Accordingly, a computer-based system 302 may include stored information 324 that varies by practice or is uniformly limited across a plurality As a result, in some embodiments, the system 300 allows practitioners to leverage information from a plurality of otherwise unassociated practitioners, patients and practices.

In accordance with one embodiment, the computer system 312 may include some portion of the stored information 324 instead of (or in combination with) the computer-based system 302. That is, each practice may selectively retain some information concerning the practitioner recommended treatment and treatment results on the computer system 312 to maintain control over that data or subset of data. They may then provide that information on an as needed basis over the network 306 to the computer-based system 302 when an evaluation and/or recommendations are to be formally prepared by the computer-based system 302. For example, the recommendations may be generated on an as requested basis. In some embodiments, the preceding is a practice based decision that can be made individually on a practice by practice basis such that a first practice has more complete information concerning their recommended treatments and treatment results along with automatic generation of supply recommendations generated proactively by the computer-based system 302. In other practices, however, the practitioners may opt to seek supplier recommendations on an as needed basis.

In accordance with any of the preceding embodiments, however, the evaluation module 322 can be employed to evaluate the practitioner recommended treatments and treatment results in view of the known effectiveness and suitability of the various products and services provided by the supplier. This data can be employed the evaluation module 322 to generate one or more supplier recommendations that are communicated over the network to one or more of the practices.

In the illustrated embodiment, the form of communication can take advantage of the Internet, and accordingly, the computer-based system 302 can employ the e-mail server 314 to provide e-mail alerts to the practitioners when a recommendation is available. The e-mail notification can provide the actual substance of the recommendation as well, for example, the body of the e-mail may include the recommendations. In addition the e-mail may include one or more attached documents including images, PDF's, and word processing documents including reports that provide the information desired by the practitioner. In accordance with some embodiments, a web server 316 can facilitate the sharing of information between the practices and the computer-based system 302. For example, the web server may provide system login access over the internet to the various practitioners and allow them to access the secure information including any of the treatment results, past recommended treatments and supplier recommendations. Further, the web server may include links to various documents that are directly accessible to the practitioner online.

In accordance with some embodiments the computer-based system 302 maintains an implementation history concerning past supplier recommendations, the adoption of the past supplier recommendations and the treatment outcome/progress that occurred following the adoption of supplier recommendations. Further, the computer-based system 302 may include stored information 324 concerning the rate of adoption by a particular practitioner or practice or type of recommended procedure provided by the computer-based system to the practitioners.

According to some embodiments, the system 300 can be employed to assist a practitioner in increasing the volume of treatments that they perform and the profitability of their practice. The system can assist practitioners in this regard because of the historical data concerning past treatment procedures can be placed in a broader business and market context, for example, when the data for a particular treatment is aggregated across a plurality of practices and when the data for a single practice is evaluated against the broader treatment industry.

Features and/or elements of the various computer-based systems described herein can be advantageously combined in various embodiments. For example, referring to FIG. 1, all or a portion of the functionality of the control system 106 can be included in the computer system 108 according to one embodiment. As another example, the computer system 108 of FIG. 1 may be included in a computer system 312 illustrated in FIG. 3.

Although some embodiments described herein concern the treatment of an aesthetic condition of a subject, the above-described embodiments can be applied in a wider variety of medical treatments such as surgical procedures including laser surgery, and eye surgery. In general, embodiments of the invention can be employed in any medical practice-based environment or any treatment environment which offers physical treatments and/or procedures to an end customer. Further, the term aesthetic condition can refer to any condition that affects an appearance of a patient. For example, an aesthetic condition may be a cosmetic skin condition or a condition caused by the bone and/or jaw structure of the patient, for example, an orthodontic condition.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system comprising:
an energy application device configured to apply energy to a treatment area of a subject;
a sensing device configured to determine the energy received by one or more regions of the treatment area; and
a user interface configured to provide a user with feedback concerning the energy received by the one or more regions of the treatment area, wherein the feedback provided to the user includes an image that illustrates the current status of the treatment.

2. The system according to claim 1, wherein the illustration includes quantitative information.

3. The system according to claim 1, wherein the illustration includes highlighting of the treatment area.

4. A computer-based method comprising acts of:
storing patient treatment data for a subject patient;
storing reference treatment data for a plurality of patients where the reference treatment data includes at least one image;
evaluating a current status of treatment for the subject patient; and
providing at least one recommended course of treatment for the subject patient based on a comparison between the current status and a result achieved in treating a similarly situated patient.

5. The method of claim 4, further comprising an act of including at least one image of the similarly situated patient with the recommendation.

6. The method of claim 5, further comprising an act of communicating the recommendation to the patient via the Internet.

7. The method of claim 5, further comprising an act of evaluating a treatment of an aesthetic condition of the subject patient.

8. A computer-based method comprising acts of:
centrally-collecting information concerning patient treatment from a plurality of practices where the information concerns recommended courses of treatment and associated results;
storing information concerning a plurality of products and services available from a supplier of treatment systems;
receiving at least one practitioner recommended treatment for a subject patient;
evaluating the recommended treatment in view of (a) the current treatment status of the subject patient, (b) centrally collected information concerning at least one similarly situated patient and (c) the plurality of products and services available from the supplier; and
providing at least one recommended change to the practitioner recommended treatment based on the act of evaluating.
